(19) United States
(12) Patent Application Publication
(10) Pub. No.: US 2006/0263350 A1
(43) Pub. Date: Nov. 23, 2006

(54) Combination antihistamine medication

(75) Inventor: Edward M. Lane, Weston, CT (US)

Correspondence Address: ROTHWELL, FIGG, ERNST & MANBECK, P.C., 1425 K STREET, N.W., SUITE 800, WASHINGTON, DC, 20005, US

(73) Assignee: Fairfield Clinical Trials LLC, Bridgeport, CT (US)

(21) Appl. No.: 11/494,599

(22) Filed: Jul. 28, 2006

Related U.S. Application Data
(63) Continuation-in-part of application No. 11/389,498, filed on Mar. 27, 2006.
Application No. 11/389,498 is a continuation of application No. PCT/US04/31380, filed on Sep. 27, 2004.
(60) Provisional application No. 60/505,920, filed on Sep. 26, 2003.

(51) Int. Cl. A61K 39/395; A61K 31/573; A61K 39/02

(52) U.S. Cl. 424/131.1; 424/236.1; 514/171

(57) ABSTRACT

The invention provides a topical pharmaceutical composition for application to the nasal or ocular mucosa which comprises (1) a pharmaceutical excipient suitable for topical administration, (2) a mucosal adjuvant, (3) an antihistamine drug and (4) a mast cell stabilizer, a non-steroidal anti-inflammatory drug, a phosphodiesterase inhibitor, an anti-IgE agent, heparin, a topical steroid or a leukotriene blocker.

### CROSS REFERENCE TO RELATED APPLICATION

This application is a continuation-in-part of prior co-pending application U.S. Ser. No. 11/389,498, filed Mar. 27, 2006, which is a continuation of International Application No. PCT/US2004/031380, filed Sep. 27, 2004, which claims priority from United States Provisional Application No. 60/505,920, filed Sep. 26, 2003. The disclosures of both the above Applications are hereby incorporated by reference in their entirety.

### BACKGROUND OF THE INVENTION

1. Technical Field

This invention is related to the field of medicine, and in particular to combined pharmaceutical compositions and methods for treatment of seasonal or perennial allergic rhinitis, or non-allergic (vasomotor) rhinitis.

2. Description of the Background Art

Seasonal allergic rhinitis (SAR), Perennial Allergic Rhinitis (PAR) and non-allergic rhinitis are inflammatory conditions of the upper respiratory system. Although avoidance of the allergen is the cornerstone of conventional therapy for allergic rhinitis, this is not always possible and does not relate to non-allergic rhinitis. Medical therapy is often added for those patients who are still symptomatic. Medical therapy traditionally has relied on systemic antihistamines taken orally, although a newer antihistamine, azelastine, is delivered by nose spray. Nasally administered steroids also are used in treating these conditions. They are particularly beneficial in preventing or dampening the allergic response. Other compounds, such as ipratropium, chromolyn, topical and systemic decongestants, leukotriene blockers such as zileuton and montelukast and systemic steroids have thus far demonstrated limited roles in therapy when used alone.

Signs and symptoms of different types of rhinitis may overlap but include nasal congestion, sneezing, watery rhinorrhea, post-nasal drip, Eustachian tube dysfunction, pharyngitis, cough, and ocular symptoms, particularly itchy eyes. Allergens which commonly cause symptoms include pollen, animal dander, mold, dust and dust mites, and others. Rhinitis results from other causes, mainly viral, but also in response to environmental exposure such as to toxic chemicals and tobacco smoke. Bacterial infections, fungal infections, parasites, collagen vascular diseases, sarcoidosis, Wegener's granulomatosis, and lethal midline granuloma occur much less frequently. The diagnosis of SAR or PAR can be confirmed by allergy testing, either skin testing (e.g. a prick test) or by serum assay (e.g. RAST). Usually however, therapy is begun empirically based on a patient's constellation of symptoms rather than the exact etiology.

Rhinitis causes considerable discomfort and morbidity associated with symptoms that affect work or school performance and cause significant changes in Quality of Life (QOL) scales in those who suffer from it. Although allergic and non-allergic rhinitis are quite common, and various treatments have been and are available, satisfactory medications for treatment have been lacking in the art.

### SUMMARY OF THE INVENTION

Accordingly, this invention provides, in one embodiment, a topical pharmaceutical composition for application to the nasal or ocular mucosa which comprises a pharmaceutical excipient suitable for topical administration, a mucosal adjuvant, an antihistamine drug and a drug composition selected from the group consisting of a mast cell stabilizer, a non-steroidal anti-inflammatory drug, a phosphodiesterase inhibitor, an anti-IgE agent, heparin, a topical steroid and a leukotriene blocker. Preferred mast cell stabilizers are cromolyn, cromoglycate, lodoxamide tromethamine, nedocromil, olopatadine and pemirolast. A preferred nonsteroidal anti-inflammatory drug is ketorolac tromethamine. A preferred phosphodiesterase inhibitor is roflumilast. Preferred anti-IgE agents are anti-IgE antibodies (Omalizumab™). Preferred topical steroids are fluticasone, beclomethasone, budesonide, triamcinolone and mometasone. Preferred leukotriene blockers or modifiers are olopatadine, zileuton, pranlukast, zafirlukast and montelukast. Preferred mucosal adjuvants are a Vibrio cholerae toxin, chitosan, microparticles, polymeric lamellar substrate particles, synthetic biomimetic super molecular Biovector™, an absorption enhancer, a CpG oligodeoxynucleotide, phenylpropanolamine, supersaturated potassium iodide (SSKI)), a chaotropic agent, a bioadhesive agent and a mucolytic agent. Most preferred mucosal adjuvants are a Vibrio cholerae toxin, chitosan, poly(lactide co-glycolide) microparticles, a CpG oligodeoxynucleotide and a bioadhesive agent.

In another embodiment, the invention provides a method of treatment of allergic or non-allergic rhinitis which comprises administering to the nasal or ocular mucosa a topical pharmaceutical composition as described above.

### DETAILED DESCRIPTION OF THE PREFERRED EMBODIMENTS

Symptoms of allergic rhinitis result from exposure to triggering antigens in a sensitized individual. These antigens interact with IgE, bound to the surface of mast cells in the nasal mucosa (or to circulating basophils) via the high affinity IgE receptor. Recognition and binding of the antigen by the IgE activates these cells, which release mediators, including histamine and leukotrienes, and cytokines that attract inflammatory cells. Allergic rhinitis is associated with early symptoms (early phase symptoms primarily involve nasal itching but also may include sneezing and congestion) and late symptoms (late phase symptoms are marked primarily by nasal congestion).

Intranasal and intraocular corticosteroids exert a range of effects that inhibit mucosal inflammation, including (1) reducing inflammatory cell infiltration, (2) decreasing the number of basophils, eosinophils, neutrophils and mast cells in the nasal passages and their secretions, (3) reducing release of inflammatory signals from cells, (4) decreasing mucus production, (5) vasoconstriction and (6) reducing edema. Antihistamines block histamine receptors in the mucous gland and mucosal vasculature, which prevents histamine from exerting its effects in the early phases of allergic rhinitis. Leukotriene receptor antagonists (also known as leukotriene blockers) block the action of leukotrienes on target cells which occurs in the late phases of allergic rhinitis. Blockade of leukotrienes results in decreased vasodilation, vascular permeability, and mucous secretion, and therefore decreased nasal congestion. Anti-IgE agents act early in the allergic-inflammatory process to block IgE from causing the initial reaction that can lead to symptoms of SAR or PAR.

Non-allergic rhinitis involves sporadic or persistent nasal symptoms not resulting from actions of IgE. This syndrome is diagnosed when no allergen can be detected through diagnostic testing and no other obvious cause is evident. Typical symptoms are similar to those discussed above for SAR, such as nasal itching, rhinorrhea, nasal obstruction, and occasionally, change or loss of sense of smell.

Because the cause of both allergic and nonallergic (vasomotor) rhinitis and conjunctivitis is multifactorial, the invention acts in concert at different points in the allergic cascade at the same time to improve treatment efficacy. Treatment according to the invention therefore can lead to increased efficacy, with fewer side effects.

While most SAR and PAR patients with mild symptoms use only one therapeutic agent at a time, a significant number with moderate to severe symptomology do not respond adequately to these regimens. Such patients require a combination of therapy including an antihistamine and, for example, a nasally active steroid and/or a leukotriene blocker, which is provided by an embodiment of this invention. Mucosal inflammation and swelling caused by the body's response to the presence of the allergen(s) can prevent topical medications such as spray antihistamines from reaching the affected area or reaching the affected area in adequate amounts or concentrations. Antihistamines are known to be ineffective in relieving nasal obstruction. This invention can overcome this problem in the art by, in one embodiment, combining a topical nasally active steroid, a non-steroidal antiinflammatory agent, a mast cell stabilizer or other drugs as listed below, together with a topical antihistamine. The addition of a steroid drug reduces the inflammatory response and renders the topical antihistamine more efficacious, providing a greatly improved therapeutic effect, whether administered nasally or by another route, such as ocularly. In addition to this combination of agents active in treating allergic rhinitis, the compositions of the invention also optionally contain a mucosal adjuvant, which enhances the ability of the active agents to exert their effects.

Decongestants for oral or nasal administration are known in the art and have been used in combination with antihistamines for treatment of allergic rhinitis. These agents, when applied nasally, usually are effective only for short term use. For long-term use, decongestants generally are delivered orally and are somewhat less effective but less susceptible to “rebound” vasodilation after cessation of treatment.

Corticosteroids have been useful as monotherapy for mild to moderate allergic rhinitis, but generally require several days to reach maximum effect. These agents are most effective in monotherapy when treatment is begun one to two weeks prior to exposure to the allergen, for example prior to the appearance of seasonal pollen-related symptoms. Unfortunately, it is not always possible to predict when exposure to the allergen will occur. Oral corticosteroids are not recommended for treatment of ordinary SAR or PAR and are reserved for the most intractable cases.

Mast cell stabilizing compounds such as cromolyn can be effective in treating established allergic reactions, but require frequent dosing and continuous usage over a period of time to achieve the desired effect. In general, these agents are considered not as efficacious as either antihistamines or nasal corticosteroids.

As the term is used herein, “mucosal adjuvant” refers to a compound that increases the absorption and/or effectiveness of an active drug compound when applied topically to a mucosal area to be treated, for example the nasal mucosa, the ocular mucosa, and other tissues as discussed herein. Mucosal adjuvants therefore are compounds that stimulate or increase local action of the active compound. These compounds also may increase the systemic absorption of the active compound(s), but need not do so and preferably do not. Similar to the way the term is used with respect to vaccine adjuvants, mucosal adjuvants may exert their effects in several different ways, and similar also to vaccine adjuvants. The exact mechanism of adjuvant action may not be known.

Thus, mucosal adjuvants may operate by creating a “depot” effect, wherein the drug or active composition(s) are held in a site where they may be absorbed by or into the tissue to a greater extent or over a longer period of time, for example because of a bioadhesive effect. Mucosal adjuvants also may exert their enhancing effects by affecting the mucosal barrier (“mucus blanket”) which covers the tissues to be treated, for example by reducing mucus production or thinning or dissolving the mucus. Therefore, mucolytic agents can act as mucosal adjuvants. Another mechanism whereby a mucosal adjuvant can exert its effect is by modifying the tight junctions between cells in the mucosal tissue to be treated so that the active compounds have better access to the tissue and optionally better systemic absorption. In addition, a mucosal adjuvant may alter the ciliary action at the surface of some mucosal tissues which otherwise would sweep away the drug composition prior to absorption by the cells or tissue. A mucosal adjuvant differs from certain vaccine adjuvants, which are designed to produce an irritation or inflammatory response, since an object of embodiments of the invention is to reduce inflammation of the mucosal tissue which may be caused by allergic responses. Mucosal adjuvants for use in this invention therefore preferably are minimally irritating to the mucosal tissue to which they are applied.

Preferred mucosal adjuvants include but are not limited to toxins from the bacterium Vibrio cholerae (cholera toxin, cholera toxin A subunit, cholera toxin B subunit), chitosan, poly(lactide co-glycolide) (PLG) microparticles, polymeric lamellar substrate particles (PLSP), synthetic biomimetic super molecular Biovector™ (SMBV), absorption enhancers (e.g., cyclodextrin, glycols and the like) bioadhesive agents (e.g., carbopol, celluloses, starch, dextran and the like), and CpG oligodeoxynucleotides. Additional examples of mucosal adjuvants include, but are not limited to phenylpropanolamine, sodium or potassium iodide (for example supersaturated potassium iodide (SSKI)), sodium thiocyanate, N-acetylcysteine, dithiothreitol, urea or guanidine hydrochloride, guaifenesin, antimony potassium tartrate, squill, ipecac syrup extract, terpin hydrate, tyloxapol or certain proteases, for example trypsin, chymotrypsin and the like, which reduce the thickness (viscosity) of mucus.

Any mucosally compatible chaotropic agent or other compound that can reduce the viscoelastic consistency of mucus can serve as a mucosal adjuvant. Pharmaceutically compatible mucolytic agents are described in the art, for example in Remington's Pharmaceutical Sciences, 20th edition, Mack Publishing Co., 2000, the disclosures of which are hereby incorporated by reference. Formulations wherein the drug compounds are provided in liposomes, microparticles or in any other formulation that more successfully penetrates the natural barriers of mucosal tissues or acts as a depot also can act as a mucosal adjuvant, for example poly(lactide co-glycolide) microparticles, polymeric lamellar substrate particles, synthetic biomimetic supra molecular Biovector™ (SMBV) and the like, for example may be used as a mucosal adjuvant.

The invention provides, in different embodiments, combination treatments and compositions which can intervene with the allergic cascade at multiple points and provide superior relief of symptoms. In addition, combination medications which contain each pharmaceutical in a single pharmaceutical preparation or dosage form for topical delivery provide improved simplicity in dosing, improved patient compliance and significant cost savings to both the patient and the patient's insurance carrier.

The invention provides an embodiment comprising a combination medication for topical administration, including nasal, ocular or otic administration, and sublingual, transdermal and trans-buccal administration in some embodiments. Medications according to the invention contain an antihistamine drug, for example astemizole, azelastine, brompheniramine, chlorpheniramine, cetirizine, clemastine, desloratidine, dexbrompheniramine, diphenhydramine, doxylamine, ebastine, emedastine, epinastine, fexofenadine, hydroxyzine, ketotifen, levocabastine, levocetirizine, loratidine, mequitazine, mizolastine, olopatadine, oxatomide, phenindamine, pheniramine, pyrilamine, terfenidine, triprolidine, or any combination or active isomer or prodrug thereof, a mucosal adjuvant, and at least one of the following classes of pharmaceutical products, in a single administrable dose:
1. a topical steroid, for example fluticasone, beclomethasone, budesonide, triamcinolone, mometasone;
2. a leukotriene blocker or modifier, for example zileuton, pranlukast, zafirlukast, montelukast;
3. a mast cell stabilizer, for example cromolyn, cromoglycate, lodoxamide tromethamine, pemirolast, olopatadine;
4. a nonsteroidal anti-inflammatory drug, for example ketorolac tromethamine;
5. a decongestant, for example phenylpropolamine, pseudoephedrine, oxymetazoline;
6. a phosphodiesterase inhibitor, for example roflumilast;
7. an anti-IgE agent, for example anti-IgE antibodies, omalizumab;
8. an anticholinergic agent, for example tiotropium, ipratropium; or
9. any drug known to be useful in the treatment of allergic or non-allergic rhinitis, for example heparin, capsaicin, guaifenesin; and
10. optionally, a mucosal adjuvant.

The combination medication preferably is in the form of an aqueous solution or suspension, with a pharmaceutically acceptable carrier such as sterile water or saline, which contains effective amounts of both an antihistamine and a second drug such as a nasally active steroid or other drug as listed above, and optionally, a mucosal adjuvant. Such medications may be delivered conveniently by a pump-actuated nose sprayer or by a medicine dropper or dropper bottle to the nasal passages, the eye(s) or the ear(s). Alternative methods of administration include but are not limited to aerosolizers, nebulizers (such as used with SinuNeb®), douching apparatuses (such as Netti pots™), compressed gas actuators (such as those used with Beconase® or Vancenase®, dry powder (such as used for Advair®, Pulmicort® or Nasacort AQ®) to be inhaled nasally or delivered to the ear canal), and atomizers. Other dosage forms for topical administration are known in the art and are suitable for use with the invention, including but not limited to lotions, creams, and so on. Any of the formulations may contain additional pharmaceutical excipients such as buffers, fragrances, diluents, preservatives etc. as are known in the art. Additional active ingredients also optionally are present, such as a demulcent, an antiseptic, a local anesthetic or numbing agent, and the like.

Any of the known antihistamines and any pharmaceutically acceptable salts thereof, which are effective when applied topically to the nasal mucosa, eyes or ear canal in an aqueous or other mucosally compatible solution, suspension or other topical preparation, may be used in the inventive compositions. Preferred antihistamines for use with the invention include azelatine, cetirizine, desloratidine, fexofenadine, olopatadine or any pharmaceutically acceptable salt thereof, however any of the antihistamines listed in Table II or their pharmaceutically acceptable salts, enantiomers, active metabolites or prodrugs also may be used. Any of these antihistamine compounds can be combined with, for example, any known steroid that is active when applied topically to the mucosa (see, for example, Table III) in the presence or absence of a suitable mucosal adjuvant as described herein, or any of the other drug classes listed herein.

Suitable dosages of antihistamine for nasal or other application can be easily determined by the skilled clinician. The known antihistamine azelatine, which is administered nasally, serves as a guide for determining a suitable dose for any other antihistamines for topical nasal administration. Therefore, combination compositions generally contain about 1 μg to about 10 mg, preferably about 10 μg to about 250 μg and most preferably about 100 μg to about 150 μg (per metered dose) antihistamine compound. Clinicians generally have experience with antihistamine compounds for oral dosing and can easily determine a suitable dose for use in combination with any of the known topically active steroids, leukotriene blockers, mast cell stabilizers, etc. Appropriate doses for the nasally active steroid in the inventive combination medication can follow current FDA guidelines and are easily determined by the skilled clinician. Generally, combination compositions of the invention contain about 1 μg to about 1 mg, preferably about 30 μg to about 80 μg, and most preferably about 45 μg to about 65 μg steroid compound per metered dose.

In compositions of the invention that contain a mucosal adjuvant, the adjuvant generally is present in an effective amount, which is any amount sufficient to provide the enhancing or stimulating effect of the adjuvant in question. For example, an emulsion formulation in which the emulsifying agent is acting as the adjuvant would contain sufficient emulsifying agent to form the desired emulsion. Microparticle adjuvants would be present in such an amount sufficient to contain the active drug of the embodiment and release an effective dose of the active drug to the mucosal layer to be treated. Compounds that act by affecting tight junction barriers are present in an amount effective to perform that function sufficiently well to increase permeation of the active ingredients and thereby their effectiveness. Chaotropes, mucolytic agents and the like, which exert their effect by reducing or thinning mucus are present in amounts sufficient to perform that function.

The compounds as discussed above are known per se in the art and are familiar to those of skill in the art. Therefore, amounts which are effective to achieve the effects as discussed above, and others which can be discerned by those of skill in the art based on general knowledge and the guidance herein, are either known or can be easily discovered or modified by the skilled person. However, examples of appropriate amounts of adjuvant to form a part of embodiments of this invention are provided below.

TABLE I
Exemplary Adjuvant Amounts.

| Type of | | Exemplary Amounts |
|---|---|---|
| Adjuvant | Examples | (Preferred Amount) |
| Emulsion | PEG stearate ester/cetyl | 3-10% by wt |
| | stearilic alcohol, e.g. | (4% by wt) |
| | Emulpharma ® 200 | |
| Depot | carboxymethylcellulose | 1-10% by wt |
| | sodium | (4% by wt) |
| Bioadhesive | methyl vinyl ether/maleic | 1-10% by wt |
| | anhydride copolymer, e.g. | (8% by wt) |
| | Gantrez ® AN-139 | |
| Mucolytic | Guaifenesin | 0.1 μg-1.0 mg |
| | | (1.0 μg) |
| Ciliary Action | benzalkonium chloride | 0.01 μg-1.0 mg |
| Affector | | (1.0 μg) |
| Tight Junction | anti-occludin antibodies | 0.1-10 nM/mL |
| Modifier | anti-ZO-1 antibodies | (1.0 nM/mL) |

TABLE II
Selected Exemplary Antihistamine Compounds.
Generic name

loratadine
desloratidine
fexofenadine
cetirizine
azelatine
azatadine
clemastine
olopatadine
brompheniramine
chlorpheniramine
dexbrompheniramine
diphenhydramine
doxylamine
phenindamine
pheniramine
pyrilamine
triprolidine
levocabastine
acrivastine
carbmoxamine
dexchlorpheniramine
promethazine
trimeprazine
methdilazine
hydroxyzine
rocastine
tripelennamine
meclizine
tripolidine
cyproheptadine
methscopolamine
phenylpropanolamine

TABLE III
Exemplary Steroid Compounds.
Generic Name

fluticasone
mometasone
beclomethasone
triamcinolone
budesonide
flunisolide
dexamethasone

### EXAMPLES

Exemplary Combination Medications.

TABLE IV
Preferred Medications.

| Example | Antihistamine | Other | Adjuvant |
|---|---|---|---|
| 1 | desloratidine | mometasone | chitosan |
| 2 | loratidine | mometasone | CpG |
| | | | oligodeoxynucleotide |
| 3 | fexofenadine | triamcinolone | poly(lactide co- |
| | | | glycolide) |
| | | | microparticles |
| 4 | cetirizine | fluticasone | SMBV |
| 5 | azelatine | budesonide | Emulpharma ® 200 |
| 6 | olopatadine | montelukast | Vibrio cholerae toxin |
| 7 | levocabastine | fluticasone | poly(lactide co- |
| | | | glycolide) |
| | | | microparticles |
| 8 | desloratidine | zileuton | Guaifenesin |
| 9 | loratidine | olopatadine | carboxymethylcellulose |
| | | | sodium |
| 10 | fexofenadine | zafirlukast | chitosan |
| 11 | cetirizine | montelukast | SMBV |
| 12 | azelatine | cromolyn | Vibrio cholerae toxin, |
| | | | subunit B |
| 13 | olopatadine | budesonide | benzalkonium |
| | | | chloride |
| 14 | levocabastine | guaiafenesin | poly(lactide |
| | | | co-glycolide) |
| | | | microparticles |
| 15 | desloratidine | lodoxamide | CpG |
| | | tromethamine | oligodeoxynucleotide |
| 16 | loratidine | nedocromil | anti-ZO-1 |
| | | | antibodies |
| 17 | fexofenadine | pemirolast | SSKI |
| 18 | cetirizine | ketorolac | Vibrio cholerae toxin |
| | | tromethamine | subunit A |
| 19 | azelatine | roflumilast | cellulose |
| 20 | olopatadine | guaiafenesin | CpG |
| | | | oligodeoxynucleotide |
| 21 | levocabastine | beclomethasone | mucolytic |
| 22 | desloratidine | omalizumab | SMBV |
| 23 | loratidine | anti-IgE | Vibrio cholerae toxin |
| 24 | fexofenadine | heparin | Gantrez ® AN-139 |
| 25 | cetirizine | ipratropium | poly(lactide |
| | | bromide | co-glycolide) |
| | | | microparticles |
| 26 | azelatine | nedocromil | chitosan |
| 27 | olopatadine | cromolyn | anti-occludin |
| | | | antibodies |
| 28 | desloratidine | cromoglycate | CpG |
| | | | oligodeoxynucleotide |
| 29 | fexofenadine | beclomethasone | Emulpharma ® 200 |

1. A topical pharmaceutical composition for application to the nasal or ocular mucosa which comprises:
(a) a pharmaceutical excipient suitable for topical administration,
(b) a mucosal adjuvant,
(c) an antihistamine drug and
(d) a drug composition selected from the group consisting of a mast cell stabilizer, a non-steroidal anti-inflammatory drug, a phosphodiesterase inhibitor, an anti-IgE agent, heparin, a topical steroid and a leukotriene blocker.

2. A topical pharmaceutical composition of claim 1 wherein said adjuvant compound is selected from the group consisting of a Vibrio cholerae toxin, chitosan, a microparticle, a polymeric lamellar substrate particle, synthetic biomimetic super molecular Biovector™, an absorption enhancer, a CpG oligodeoxynucleotide, phenylpropanolamine, supersaturated potassium iodide (SSKI)), a chaotropic agent, a bioadhesive agent and a mucolytic agent.

3. A topical pharmaceutical composition of claim 1 wherein said adjuvant compound is selected from the group consisting of a Vibrio cholerae toxin, chitosan, poly(lactide co-glycolide) microparticles, a CpG oligodeoxynucleotide and a bioadhesive agent.

4. A topical pharmaceutical composition of claim 1 wherein said mast cell stabilizer is selected from the group consisting of cromolyn, cromoglycate, lodoxamide tromethamine, nedocromil, olopatadine and pemirolast.

5. A topical pharmaceutical composition of claim 1 wherein said nonsteroidal anti-inflammatory drug is ketorolac tromethamine.

6. A topical pharmaceutical composition of claim 1 wherein said phosphodiesterase inhibitor is roflumilast.

7. A topical pharmaceutical composition of claim 1 wherein said anti-IgE agent is selected from the group consisting of an anti-IgE antibody and omalizumab.

8. A topical pharmaceutical composition of claim 1 wherein said topical steroid is selected from the group consisting of fluticasone, beclomethasone, budesonide, triamcinolone and mometasone.

9. A topical pharmaceutical composition of claim 1 wherein said leukotriene blocker is selected from the group consisting of zileuton, pranlukast, zafirlukast and montelukast.

10. A topical pharmaceutical composition of claim 1 wherein said antihistamine drug is selected from the group consisting of astemizole, azelastine, brompheniramine, chlorpheniramine, cetirizine, clemastine, desloratidine, dexbrompheniramine, diphenhydramine, doxylamine, ebastine, emedastine, epinastine, fexofenadine, hydroxyzine, ketotifen, levocabastine, levocetirizine, loratidine, mequitazine, mizolastine, olopatadine, oxatomide, phenindamine, pheniramine, pyrilamine, terfenidine, triprolidine, or any combination or active isomer or prodrug thereof.

11. A method of treating of allergic or non-allergic rhinitis which comprises administering to the nasal or ocular mucosa a topical pharmaceutical composition of claim 1.
